(19) United States
(12) Patent Application Publication
(10) Pub. No.: US 2023/0104767 A1
(43) Pub. Date: Apr. 6, 2023

(54) HEALTH AND SAFETY MANAGEMENT SYSTEM

(71) Applicant: XORIA LIMITED, Middleton, Christchurch (NL)

(72) Inventors: Raoul Joseph MACKLE, Papanui, Christchurch (NZ); Stephen Ian MANN, Northwood, Christchurch (NL); Peter James MONTGOMERY, Ohoka, Kaiapoi (NL)

(73) Assignee: XORIA LIMITED, Middleton, Christchurch (NL)

(21) Appl. No.: 17/904,853

(22) PCT Filed: Mar. 5, 2021
(86) PCT No.: PCT/IB2021/051838

§ 371 Date: Aug. 23, 2022

(30) Foreign Application Priority Data
Mar. 6, 2020 (NZ) 762420

(51) Int. Cl. G06Q 50/26

(52) U.S. Cl. CPC G06Q 50/265

(57) ABSTRACT

A health and safety management system for use in worksite or hospitality premises such as a restaurant is disclosed. The system comprises at least one source of health and safety related information of the worksite or hospitality premise and at least one at least one machine learning engine. The machine learning engine is configured to receive said health and safety related information as at least one input data. The machine learning engine is further configured analyse said at least one input data and predict at least one issue relating to health and safety at the worksite or the hospitality premises that requires attention by one or more authorised parties.

The present invention relates to a health and safety management system. More particularly but not exclusively it relates to a health and safety management system for hospitality premises such as restaurants.

### BACKGROUND OF THE INVENTION

Workplace accidents and injuries are fairly common. Business establishments, particularly, but not exclusively, in the hospitality industry, such as restaurants, may be hazardous for both the workers and customers. Accidents and injuries, particularly in the kitchen area of restaurants are common. Such mishaps can also occur in other areas e.g. dining hall, drive through, staircases etc.

To help prevent accidents or injury to customers and workers, businesses attempt to identify potential hazards that are present within the business premises. It is also a good practice to ensure that proper guideline(s) is/are followed. Taking precautions appropriately can also help the business to avoid costly litigation or liabilities. Further, it is also important to ensure that safety measures are followed at all times, and workers are attentive and observant to avoid injury to themselves and others. Therefore, accurate monitoring and recording of all hazardous incidents is necessary to help prevent such mishaps.

Many countries have legislation that requires each business to record and notify a regulator (e.g. a government regulatory body) as soon as possible after they become aware of a notifiable incident (e.g. serious injury or accident) occurring in that business. Such notification often needs to be made in the form of an incident report.

Current systems for recording and notifying such incidents or otherwise managing health and safety, often involve the use of forms that need to be filled or completed based on manual entry by human and therefore can involve a lengthy and tedious manual data entry process. As a result, notifications of incidents that may have arisen from an un-resolved hazard may be received too late and after additional incidents are caused by the same hazard. Further, it can be very difficult, if not impossible, to capture all information for all possible situations which could lead to recording information that is inaccurate and/or incomplete. Consequently, the results of any subsequent analysis using such information may be severely compromised.

Also, current systems do not provide usable interfaces and due to the tedious process of logging incidents suffer from significant under-reporting of incidents.

### OBJECT OF THE INVENTION

It is an object of the present invention to provide a health and safety management system and/or method which overcomes or at least partially ameliorates some of the abovementioned disadvantages or which at least provides the public with a useful choice.

### STATEMENTS OF INVENTION

In a first aspect, the present invention may broadly reside in a hospitality premises health and safety management system comprising:

at least one source of health and safety related information of said hospitality premises ; and

at least one machine learning engine that is configured to receive said health and safety related information as at least one input data and is further configured analyse said at least one input data and make a prediction of at least one issue relating to health and safety at said hospitality premises that requires attention by at least one authorised party.

In a second aspect, the present invention may broadly reside in a health and safety management system for a worksite (i.e., a business premises), said health and safety management system comprising:

at least one source of health and safety related information of said worksite, said at least one source of health and safety related information comprises or is a monitored dispensary apparatus to which multiple people have access, the monitored dispensary apparatus comprising a containment region inside of/within which items are stored, said items contain or are items for use in medical treatment,

wherein the monitored dispensary apparatus further comprises or is in operative communication with a monitoring system that comprises at least one detector that is configured to detect any use of said monitored dispensary apparatus,

wherein, an output of said at least one detector is said health and safety related information of said worksite, and

wherein, the said health and safety management system further comprises at least one machine learning engine that is configured to receive said health and safety related information as at least one input data and is further configured to analyse said at least one input data and make a prediction of at least one issue relating to health and safety at said workplace that requires attention by at least one authorised party.

For the invention(s) as defined in the first and/or second aspects above, the features defined by one or more of the statements below may preferably apply, as appropriate.

In one embodiment, said at least one detector is located within said containment region.

In one embodiment, said machine learning engine is configured to use a machine learning algorithm for analysing said at least one input data and make said prediction of at least one issue relating to health and safety at said workplace that requires attention by said at least one authorised party.

In one embodiment, said at least one source is configured to detect or capture a health and safety related incident, said health and safety related incident being said health and safety related information.

In one embodiment, said at least one source is a monitored dispensary apparatus.

In one embodiment, said monitored dispensary apparatus comprises:

a containment region within (inside of) which items are stored, wherein said items contain or are items for use in medical treatment,

wherein the monitored dispensary apparatus further comprises or is in communication with a monitoring system,

wherein,

said monitoring system comprises at least one detector (e.g. preferably located inside said containment region) that is able to detect any use of said monitored dispensary apparatus, and

wherein an output of said one or more detectors is said health and safety information of the hospitality premises.

In one embodiment, said monitored dispensary apparatus is a first aid kit or a first aid station.

In one embodiment, said at least one source comprises or functions as at least one input device that is a computer and that allows a user to manually input said health and safety related information.

In one embodiment, said at least one input device is selected from a smartphone, laptop, personal computer (PC), tablet or PDA.

In one embodiment, said health and safety management system further comprises at least one electronic device having a display screen and a graphical user interface (GUI) displayed in said display screen, wherein said GUI is adapted to access said health and safety related information and/or said prediction, wherein said GUI is operative on said display screen.

In one embodiment, said at least one electronic device is said at least one input device.

In one embodiment, said at least one source is an environmental sensor.

In one embodiment, said at least one source is a transaction till.

In one embodiment, said at least one source is a time clock.

In one embodiment, said at least one source is a camera.

In one embodiment, said camera is a video camera and/or a surveillance camera.

In one embodiment, said health and safety management system further comprises an alert generator for generating and sending an alert to said at least one authorised party regarding said prediction.

In one embodiment, said health and safety management system further comprises a first data processing module (data pre-processing module) that is configured to receive said health and safety related information from said at least one source as an input and process said input as a processed health and safety information (processed input data).

In one embodiment, said at least one machine learning engine is configured to receive said processed health and safety information as said at least one input data.

In one embodiment, said at least one machine learning engine is configured to generate said prediction as an output data (output information) for consideration by said at least one authorised party.

In one embodiment, said at least one machine learning engine is configured to generate said prediction as an output data (output information) for monitoring and/or analysing results from said at least one machine learning engine.

In one embodiment, said health and safety management system is configured to generate a report and insights regarding said health and safety at said hospitality premises or said worksite.

In one embodiment, said health and safety management system further comprises a second data processing module (data post-processing module) that is configured to receive said prediction and generate said report and insights regarding said health and safety at said hospitality premises or said worksite based on said prediction.

In one embodiment, said health and safety management system further comprises an evaluation metrics module that is configured to receive said prediction and output a data for monitoring and/or analysing results from said at least one machine learning engine.

In one embodiment, said at least one machine learning engine is programmed or configured to split said processed input data into at least two parts said at least two parts being a training set and testing set.

In one embodiment said at least one machine learning engine is further programmed to split said processed input data into a validation set.

In one embodiment, said worksite is a hospitality premises.

In one embodiment, said worksite is a restaurant.

In one embodiment, said restaurant is a quick service restaurant.

In one embodiment, said hospitality premises is a business having a food and/or beverage preparation area.

In one embodiment, said hospitality premises is an airline or a train.

In one embodiment, said hospitality premises is a restaurant.

In one embodiment the restaurant includes at least one of the following areas:
i. a kitchen or food cooking facility,
ii. a food preparation area
iii. a food order area,
iv. a payment transaction area
v. a food service area,
vi. a dining area for a customer,
vii. a food storage area (e.g. an area containing a fridge or a freezer or cupboards or shelves),
viii. a food dispensing area (e.g. drink and condiment dispensing area),
ix. a wash-up area,
x. a washroom area,
xi. a toilet area,
xii. kids play area,
xiii. a drive-through area,
xiv. a drive-through payment transaction area,
xv. an entrance area, and
xvi. an office/staffroom area.

In a third aspect, the present invention may broadly reside in a hospitality premises health and safety management method, said method comprising:

receiving at least one source of health and safety related information of said hospitality premises from at least one source of health and safety related information of said hospitality premises;

feeding said health and safety related information as at least one input data to at least one machine learning engine;

using said machine learning engine for analysing said at least one input data; and

making a prediction of at least one issue relating to health and safety at said hospitality premises that requires attention by at least one authorised party.

In a fourth aspect, the present invention may broadly reside in a health and safety management method for a worksite (i.e. a business premises), said health and safety management method comprising:

receiving at least one source of health and safety related information of said worksite from at least one source of health and safety related information of said worksite, said at least one source of information comprises or is a monitored dispensary apparatus to which multiple users have access, the monitored dispensary device comprising:

a containment region inside of which items are stored, wherein said items contain or are items for use in medical treatment,

wherein the monitored dispensary apparatus further comprises or is in communication with a monitoring system that comprises at least one detector (e.g. preferably located within said containment region) and is able to detect any use of said monitored dispensary apparatus, and

wherein an output of said one or more detectors is said health and safety information of said worksite, wherein the method further comprises:

feeding said health and safety related information as at least one input data to at least one machine learning engine, and,

using the machine learning engine to analyse said at least one input data and make a prediction of at least one issue relating to health and safety at said workplace that requires attention by at least one authorised party.

For the invention(s) as defined in the third and/or fourth aspects above, the features defined by one or more of the statements below may preferably apply, as appropriate.

In one embodiment, the machine learning engine is configured to implement a machine learning algorithm and the method comprises:

using said machine learning algorithm for said analysing said at least one input data and for making said prediction.

In one embodiment, said at least one source is adapted to detect or capture a health and safety related incident, said health and safety related incident being said health and safety related information.

In one embodiment, said method further comprises using said machine learning algorithm for analysing said at least one input data.

In one embodiment, said method further comprises making a prediction of at least one issue relating to health and safety at said hospitality premises that requires attention by at least one authorised party.

In one embodiment, said method further comprises generating and sending an alert to said at least one authorised party regarding said prediction.

In one embodiment, said method further comprises receiving said health and safety related information from said at least one source as an input and process said input as a processed health and safety information (or a processed input data).

In one embodiment, said method further comprises receiving said processed health and safety information as said at least one input data.

In one embodiment, said method further comprises generating a report and insights regarding said health and safety at said hospitality premises or said worksite.

In one embodiment, the method further comprises generating said prediction as an output data (output information) for consideration by said at least one authorised party.

In one embodiment, said method further comprises receiving said prediction and generating said report and insights regarding said health and safety at said hospitality premises or said worksite based on said prediction.

In one embodiment, said method further comprises generating said prediction as an output data (output information) for monitoring and/or analysing results from said at least one machine learning engine.

In one embodiment, said method further comprises splitting said processed input data into at least two parts, said at least two parts being a training set and testing set for a machine learning purpose.

In one embodiment, said method further comprises splitting said processed input data into a validation set for a machine learning purpose.

In one embodiment, said worksite is a hospitality premises.

In one embodiment, said hospitality premises is a restaurant.

In one embodiment, said worksite is a restaurant.

In one embodiment, said restaurant is a quick service restaurant.

In one embodiment, said hospitality premises is a business having at least one food and/or beverage preparation area.

In one embodiment, said hospitality premises is an airline or a train.

In one embodiment, said hospitality premises, is a restaurant.

In one embodiment the restaurant includes at least one of the following areas:
i. a kitchen or food cooking facility,
ii. a food preparation area
iii. a food order area,
iv. a payment transaction area
v. a food service area,
vi. a dining area for customer,
vii. a food storage area (e.g. an area containing a fridge or a freezer or cupboards or shelves),
viii. a food dispensing area (e.g. drink and condiment dispensing area),
ix. a wash-up area,
x. a washroom area,
xi. a toilet area,
xii. a kids play area,
xiii. a drive-through area,
xiv. a drive-through payment transaction area,
xv. an entrance area, and
xvi. an office/staffroom area

In one embodiment, said method uses said hospitality premises health and safety management system as described above.

In one embodiment, said method uses said health and safety management system as described above.

Other aspects of the invention may become apparent from the following description which is given by way of example only and with reference to the accompanying drawings.

In this specification where reference has been made to patent specifications, other external documents, or other sources of information, this is generally for the purpose of providing a context for discussing the features of the invention. Unless specifically stated otherwise, any reference to such external documents is not to be construed as an admission that such documents, or such sources of information, in any jurisdiction, are prior art, or form part of the common general knowledge in the art.

For the purpose of this specification, where method steps are described in sequence, the sequence does not necessarily mean that the steps are to be chronologically ordered in that sequence unless there is no other logical manner of interpreting the sequence.

For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations, except where expressly specified to the contrary. It is also to be understood that the specific devices illustrated in the attached drawings and described in the following description are simply exemplary embodiments of the invention. Hence, specific dimensions and other physical characteristics related to the embodiments disclosed herein are not to be considered as limiting.

It is acknowledged that the term “comprise” may, under varying jurisdictions, be attributed with either an exclusive or an inclusive meaning. For the purpose of this specification, and unless otherwise noted, the term ‘comprise’ shall have an inclusive meaning, allowing for the inclusion of not only the listed components or elements but also other non-specified components or elements. The terms ‘comprises’ or ‘comprised’ or ‘comprising’ have a similar meaning when used in relation to the system or to one or more steps in a method or process.

As used hereinbefore and hereinafter, the term “and/or” means “and” or “or”, or both.

As used hereinbefore and hereinafter, “(s)” following a noun means the plural and/or singular forms of the noun.

When used in the claims and unless stated otherwise, the word ‘for’ is to be interpreted to mean only ‘suitable for’, and not for example, specifically ‘adapted’ or ‘configured’ for the purpose that is stated.

Unless otherwise specifically stated, the term “algorithms” or “learning algorithms” or “machine learning algorithms” as used hereinbefore and hereinafter may refer to Neural networks, deep learning models and/or any other suitable machine learning algorithms.

Unless otherwise specifically stated, the term “hospitality premises” as used herein and hereinafter may mean business premises of any relevant hospitality services such as but not limited to hotels and other lodging, food and beverage establishments, aeroplanes, trains, buses, cafes, bars, businesses having at least one food and/or beverage preparation area etc.

Unless otherwise specifically stated, the word “restaurant” as used hereinbefore and hereinafter may mean a place where people pay to sit and eat foods that are prepared, cooked and/or processed and served to them, but may also include eateries or quick service restaurants (QSRs) selling food to be eaten elsewhere. The foods may be prepared, cooked and/or processed within the premises or elsewhere.

Unless otherwise specifically stated, the word “autonomous” as used hereinbefore and hereinafter may include semi-autonomous (i.e. partially autonomous requiring some human intervention for operation) or fully autonomous (i.e. not requiring any human intervention for operation).

Unless otherwise specifically noted the term “first aid kit” used hereinbefore and hereinafter may also mean “first aid station” storing first aid tools and/or equipment.

### BRIEF DESCRIPTION OF THE DRAWINGS

The invention will now be described by way of example only and with reference to the drawings in which:

FIG. 1: shows a schematic flow diagram if an exemplary embodiment of a health and safety system according to the present invention.

FIG. 2: shows an example of the monitored dispensary apparatus for use in the system of FIG. 1.

FIG. 3: shows an example of how buttons and dialog boxes may be presented in the graphical user interface (GUI) of a device for use in the system of FIG. 1.

FIGS. 4A-4C: shows an example of a video processing algorithm that may be implemented in the system of FIG. 1.

FIGS. 5A-5D: shows an example of training and calibration of the machine learning algorithm for new installations that may be used in the system of FIG. 1.

FIG. 6: shows an example of a functionality of the system of FIG. 1 when an incident occurs.

FIG. 7: shows an example of how the system of FIG. 1 may allow interaction between the manager and a repair person.

FIG. 8A: shows an example of a list of some actions and corresponding semi-automated resolutions that may be presented to the manager during the use of the system of FIG. 1.

FIG. 8B: shows an example of a step in the logic by which the system of FIG. 1 may generate the action.

FIG. 9: shows an example of visualisation on the progress of health and safety that is represented in the form of a Risk or Hazard matrix using the system of FIG. 1.

FIG. 10: is a schematic flow diagram illustrating one example of a working principle for the machine learning model that may be used in the system of FIG. 1.

FIG. 11: is a schematic flow diagram showing one example of hardware implementation and working principle of the health and safety management system of FIG. 1.

FIG. 12: is a schematic diagram showing an example of a layout of a restaurant that may be implemented in the health and safety management system of FIG. 1.

### DETAILED DESCRIPTION

It is desirable to have a health and safety management system that can identify and monitor the causes, sources and types of injuries in worksites or hospitality premises such as restaurants in order to establish and/or improve workplace safety and/or workplace safety standards of the employees. This may help to determine any breach of those standards, and/or to help in minimising workplace hazards that could result in accidents or injuries. It is also desirable to have such a system that can accurately record information and generate incident reports in order to comply with health and safety legislation.

A need exists to provide a health and safety management system that captures incidents automatically, thereby eliminating incomplete, accurate or under-reporting of incidents. A need for a system exists that triggers a number of automated algorithms which further populate data into an incidence thereby reducing filing of forms. A need also exists for an easy to use interface that adds a human element to the incident report with minimal effort.

With reference to the above drawings, a health and safety management system according to an embodiment of the present invention is generally indicated by the numeral 100.

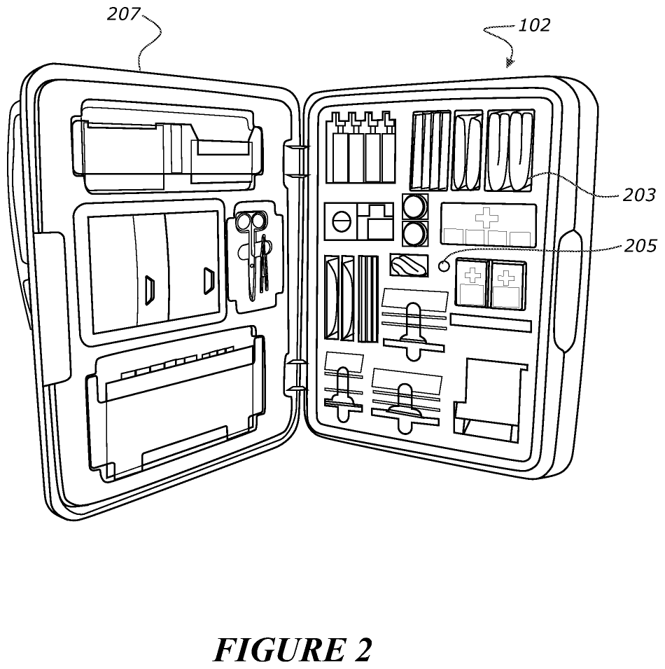

As shown, in this example, the health and safety management system 100 comprises at least one monitored dispensary apparatus 102. An example of the monitored dispensary apparatus 102 is shown in FIG. 2.

As shown, the monitored dispensary apparatus 102 (hereinafter referred to as first aid kit 102) to which multiple users may have access. The first aid kit 102 has a containment region 203 inside of/within which items containing or items for use in medical treatment (e.g. first aid equipment) may be stored. The first aid kit 102 comprises or is in an operative communication with a monitoring system having one or more detectors such as but not limited to an optical sensor/camera, e.g. camera 205. Using one or more detectors, the monitoring system captures useful information regarding the use of or pattern of use of the first aid kit 102, and such captured information is useful for creating a context of a health and safety incident. The first aid kit 102 may be the medical supplies cabinets/containers as described in WO2016/081352 and WO2019/064244, the entirety of which is incorporated herein by reference.

As mentioned above, the first aid kit 102 captures useful information regarding the use of or patterns of use of the first aid kit, and such captured information may be useful for creating a context of a health and safety incident. Example of such captured information may include a number of products used, a number of times a particular product is used, a number of times the door 207 of the first aid kit 102 is opened etc. Such captured information may be used for creating training sets for Artificial Intelligence (AI)/machine learning to predict the types of incidents (first aid incidents) that may happen. Such captured information may be used for creating testing sets and/or validity sets for use in machine learning. Such captured information may also populate the health and safety application with the prediction. Such patterns may also be used to inform replenishment supply chain optimisations, by predicting use of stock (and not over-stocking a warehouse). Therefore, the first aid kit functions as a source of health and safety related information for the machine learning algorithm.

There may be other and/or additional source(s) of health and safety related information.

As shown, the health and safety management system 100 may comprise one or more input devices (computers with display screens) such as a smartphone 104 and/or a PC 106. Other non-limiting examples of input devices that may be used include a tablet, a laptop, a PDA etc. Data gathering may be triggered via the smartphone 104, or PC 106. The programme or applications in the input device may be used to send a photo, or an SMS, email or other form of an alert, along with location and time data to instigate gathering of the information. For example, where a person such as a staff member notices a hazard, or risk, or harm that has not resulted in a first aid event, the staff member may use their input device such as smartphone 104 and/or PC 106 to activate the reporting. Similarly, the smartphone 104 and/or PC 106 can also be used to request for a timeframe the incident occurred in order to gather statistics. The staff member may input an approximate timeframe the incident occurred.

The health and safety management system 100 may comprise cameras 108a, 108b, 108c which may be surveillance cameras or video cameras. The cameras 108a, 108b, 108c may communicate with surveillance system 109 which may comprise a database 109a and a video processing means 109b to process video or footages.

The health and safety management system 100 may be used to automatically detect incident via a machine learning engine that may use/implement a machine learning algorithm. For example, as the system is used more, the video or footages that are captured and gathered and verified by users as incidents (and invalidated as not incidents) may be used to train a machine learning algorithm to detect irregular behaviour and instigate reporting for further analysis. Similarly, a sound recording may be used to further enhance the automatic detection by analysing voice data for characteristic words, or emotion, or sounds (e.g. breaking glass, a clattering of something dropped etc). Using multiple sound recordings on various cameras 108a, 108b, 108c may allow for the use of noise cancellations, or directional recordings that reduce background or unwanted noise thereby enhancing the incident detection. Other relevant data may include streamed data, voice recognition data etc.

The health and safety management system 100 may be used to align visual feeds from cameras 108a, 108b, 108c to isolate where an event occurred. For example, when the door 207 of the first aid kit 102 is open, a video stream may be fed into facial recognition, and still -frames may be captured of the best facial statistics, showing the person(s) using the first aid kit 102. An algorithm may use the facial data to determine the main person(s) involved in using the first aid kit 102 distinct from background observers and the main person(s) may be the person(s) of interest. The camera(s) 108a, 108b, 108c may be strategically located, so that the size of the face relative to other objects can give a distance and angle from the camera(s) 108a, 108b, 108c, precisely locating them, at an exact time and place in relation to the first aid kit 102. The first aid kit 102 may also comprise its own camera 205. The location of the main person(s) may be used to obtain a video of the first aid kit 102 at the time of the incident. An algorithm may trace the main person(s) back in time either a fixed time or until something triggers a stop to the algorithm. If the person of interest changes the coverage of one camera, then other camera(s) may be interrogated. The video or footage from the camera(s) 108a, 108b, 108c may be used in the health and safety application (which may be a mobile app, or any other suitable software program or application) installed in the smartphone 104 or PC 106 to allow a human user to confirm where the incident occurred by marking the frame. The health and safety application or program may then collect metrics of the incident, the number of people in the vicinity, zone, etc.

As shown, the health and safety management system 100 may further comprise one or more additional input devices (computers with display screens) such as a smartphone 110 and/or a PC 112. Other non-limiting examples of additional input devices that may be used include a tablet, a data input kiosk, a laptop, a PDA etc. These additional input devices may have health and safety application (which may be a mobile app, or any other suitable software program or application) installed on them. The health and safety application may run on any suitable operating system such as iOS®, Android® etc. Similarly, the health and safety application may also run on any suitable operating system such as Windows®, Mac OS®, Linux® etc. Alternatively, or in addition to running in the additional input devices, the health and safety application may equally run on the input devices such as smartphone 104 and/or PC 106.

The health and safety application may be populated with incidents in real-time as the incidents occur. For example, a manager and/or other staff may get notification of incidents that need triaging. The manager and/or staff may be presented with all the captured images and items of the incident. The manager/staff then may triage data, using context-sensitive buttons and dialog boxes presented in the graphical user interface (GUI) and that minimises unnecessary data collection that is not relevant. Feedback from the health and safety application may be routed with an intelligent workflow to the right person at the right level of escalation. E.g. if a customer is involved in an incident, the health and safety application may be accessible by the right person in the customer service team.

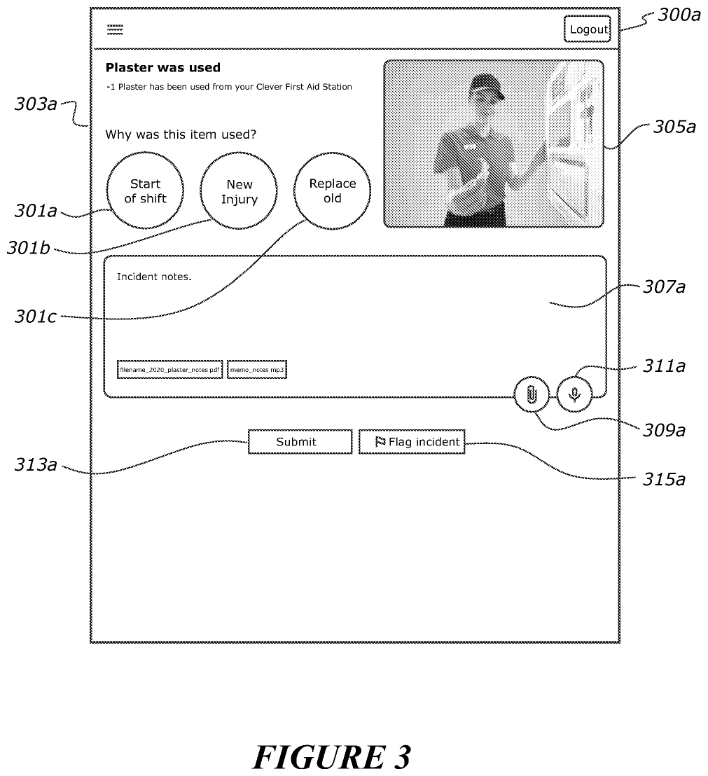

FIG. 3 shows an example of how buttons and dialog boxes may be presented in the graphical user interface (GUI) to minimise unnecessary data collection that is not relevant. As shown in FIG. 3, three buttons 301a, 301b, 301c, and dialog boxes 303a may be presented in the graphical user interface (GUI) 300a. In the example shown in FIG. 3, it is detected that a plaster has been taken from the first aid kit 102. A machine learning algorithm may detect the staff member involved (e.g. a person involved in taking the plaster from the first aid kit 102, which could be the person injured). So, the health and safety application (which may include any incident reporting software) running on smartphone 102 and/or PC 112 may be populated with the photo 305a and item (in this case a plaster) taken. Although not shown in FIG. 3, the time when the photo and item were taken may also be populated. The health and safety application or other incident reporting software may be running on the computer (e.g. smartphone) of the staff member involved and the information as shown in FIG. 3 may be presented in the computer of that user. Depending upon which device the information is sent to, the manager and/or the staff involved may then manually select the appropriate button to log or triage the incident. The user may be presented with an option to add additional notes in the field 307a. The user may also attach files using the attachment button 309a to the incident note. The user may also be presented with an option to enter an incident report using voice commands (see speech record button 311a). Once completed, the user may then click on the ‘Submit’ button 313a to complete the data input/data logging process. The user may also click on ‘Flag incident’ button 315a which may then send notify another person regarding the incident, e.g. a manager.

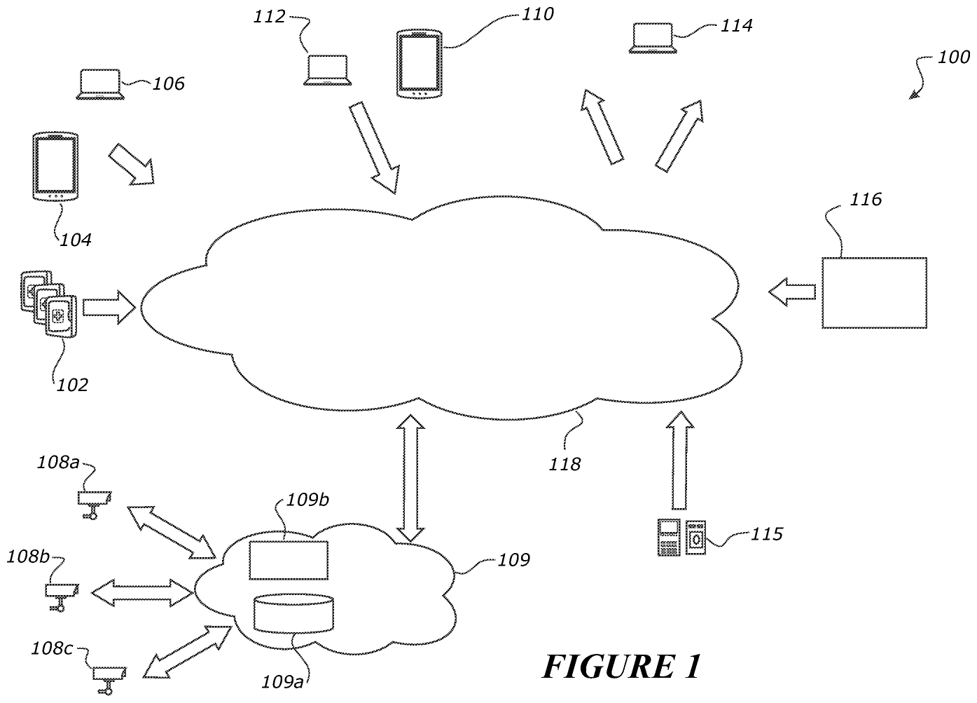

Reverting back to FIG. 1, the zone and time of the incident may be used to collect information from other sources 115, 116. These sources may provide information such as but not limited to how busy the restaurant is, how many cars were in the drive-through, last time an area was cleaned, time since the start of the shift, crowding in the area where the incident occurred. Non-limiting examples of these other sources 115, 116 may include time clocks for shift data, transaction counters from tills, queue counters for drive-through, environmental sensors for measuring CO2, humidity, Volatile Organic Compounds (VOCs) etc. It can be appreciated that some of the information such as how busy the restaurant is can also be derived from video feeds from cameras 108a, 108b, 108c, to get real-time queues/business metrics.

All sources of health and safety related information may be used to populate a health and safety incident report with factors that may be relevant to an investigation and/or further investigation. Document sources may also be retrieved from email accounts etc. to add post-incident reports from external parties, e.g. doctors, insurers, government regulatory bodies etc. The machine learning algorithm may collate the information in the rank of importance. A user (e.g. staff member) may change the order, eliminate irrelevant information, or add information that is missed. The health and safety management system 100 may also pull in data from past incidents, e.g. other incidents that occurred in the area, and are same or similar type of incident, e.g. a cut or a fall. The results of the manager of staff member validating the data may be used to train the machine learning algorithm and improve the prediction of the machine learning algorithm.

The machine learning algorithm may run on a machine learning engine, which will be described later in more detail.

These sources of information may be used to analyse metrics of health and safety usage, e.g. the number of events across all premises, e.g. restaurants normalised by business, or time of day, shift size, etc.

Statistical algorithms such as principal-component-analysis, manifold learning, classifiers, neural network-based algorithms, and any other known data-science algorithms that a skilled person may consider to be suitable may be used to obtain insights.

Metrics may be presented in a computer(s) 114 (e.g. laptop, PC, smartphone, PDA, tablet etc) in the form of meaningful reports or insights for the staff and/or any third party such as a regulator, insurer etc with minimal human input. Instead of or in addition to the computer(s) 114, the metrics may be presented in input devices 104, 106, 110, 112 in the form of meaningful reports.

All devices 102, 104, 106, 112, 110, 114, 115, 108a, 108b, 108c, 109 may communicate data/information using a cloud or cloud-based solution/application 118. Similarly, video surveillance system 118 may also communicate data/information using a cloud or cloud-based solution/application 118.

Some example of technical implementation and operation of the devices will now be described.

The health and safety management system 100 may make use of a video surveillance system of the customers, local servers and storage to minimise the data bandwidth and increase the privacy of information for customers. One example of video processing algorithms that may be implemented is illustrated in the flow diagrams of

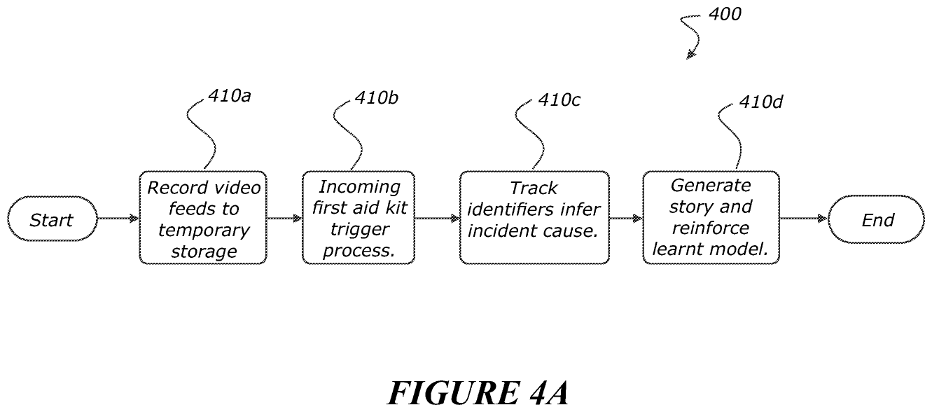

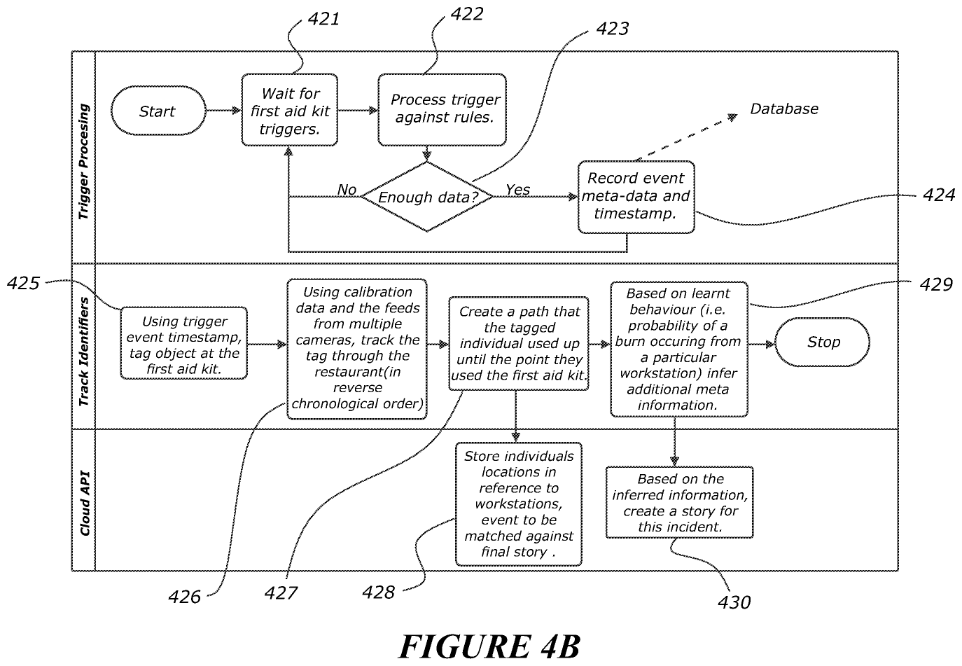

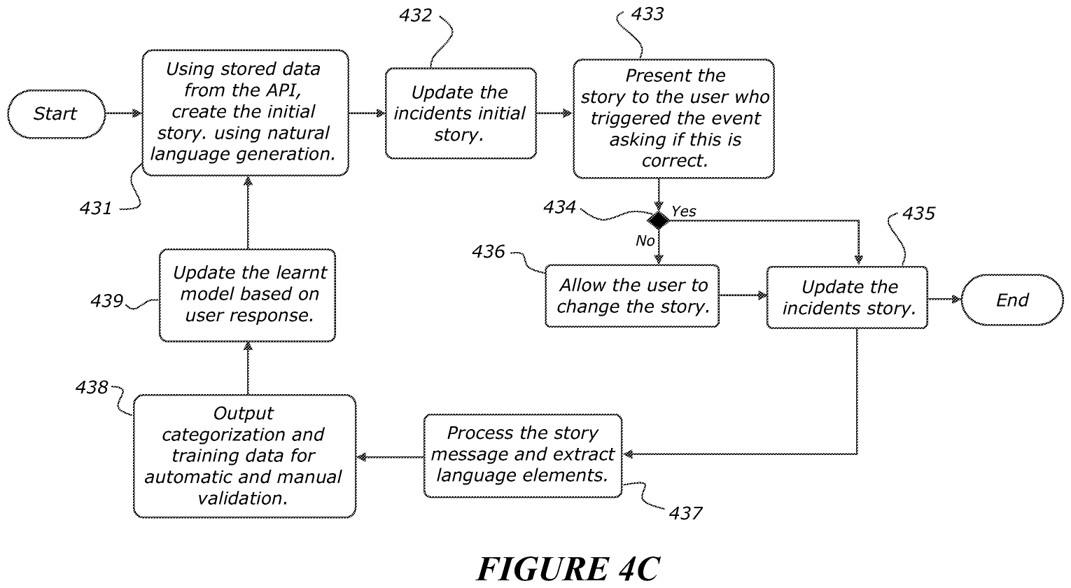

FIG. 4A-4C.

As illustrated in FIG. 4A, the video processing algorithm 400 may involve:
recording video feeds to temporary storage (step 410a);
trigger processing (step 410b);
tracking identifier (step 410c); and
generating a story and reinforcing learnt model (step 410d).

An example of a working principle of steps 410b and 401c is illustrated in the flow diagram of FIG. 4B and an example of a working principle of step 401d is illustrated in the flow diagram of FIG. 4C which is self-explanatory.

In the example shown in FIG. 4B, the trigger processing step 410b involves waiting for first aid kit 102 to trigger (step 421) and when such triggering occurs the next step (step 422) is to process triggers against rules. A check is then made if there is enough data or not (step 423). If there is enough data, then the next step is to record event meta data and timestamp in the database (step 424). However, if there is not enough data then the process reverts back to step 421.

Similarly, in the example, as shown in FIG. 4B, the step 410c of track identifier involves:
tagging object at the first aid kit 102 using a trigger event timestamp (step 425)
tracking the tag through the premises, e.g. restaurant (in reverse chronological order) using calibration data and the feeds from multiple cameras (step 426)
creating a path that the tagged individual used up until the point they used the first aid kit (step 427). This step may then further involve:
   storing individuals' locations in reference to workstations, an event to be matched against the final story. This may be done using cloud API (step 428)
inferring additional meta information based on learnt behaviour (i.e. probability of an incident such as a burn) occurring from a particular workstation) (step 429). This step may then further involve:
   creating the story of the incident, based on the inferred information. This may be done using a cloud API (step 430).

In the example shown in FIG. 4C, the step for generating story and reinforcing learnt model involve:
Creating the initial story using stored data from the API. This step may involve using natural language generation (step 431).
Updating the incidents initial story (step 432)
Presenting the story to the user which triggered the event by asking if the story is correct (step 433)
Checking if the story is correct or incorrect or inaccurate (step 434)
Updating the incident story if the story is correct (step 435) otherwise allowing the user to change the story if the story is incorrect or inaccurate (step 436).

After updating the incidents story, the process may further involve the steps of:
Processing the story messages and extracting the language elements—(step 437)
Outputting categorisation and training data for automatic and manual validation (step 438)
Updating the learnt model based on the user response (step 439)
Returning to the step of creating the initial story using stored data from the API—i.e. reverting to step 431.

The installation and calibration of the health and safety management system 100 may occur via machine learning algorithms from past and present installations.

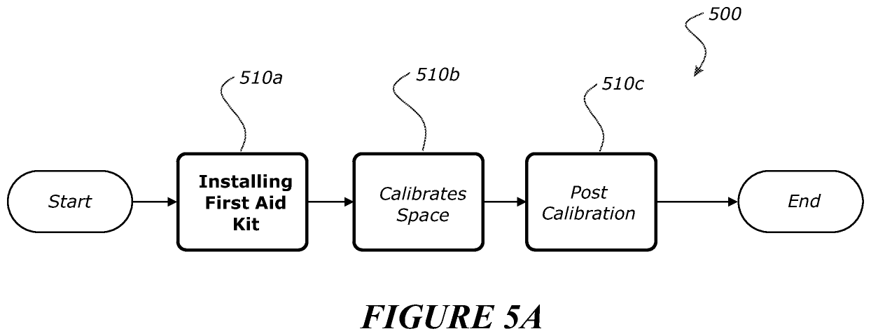

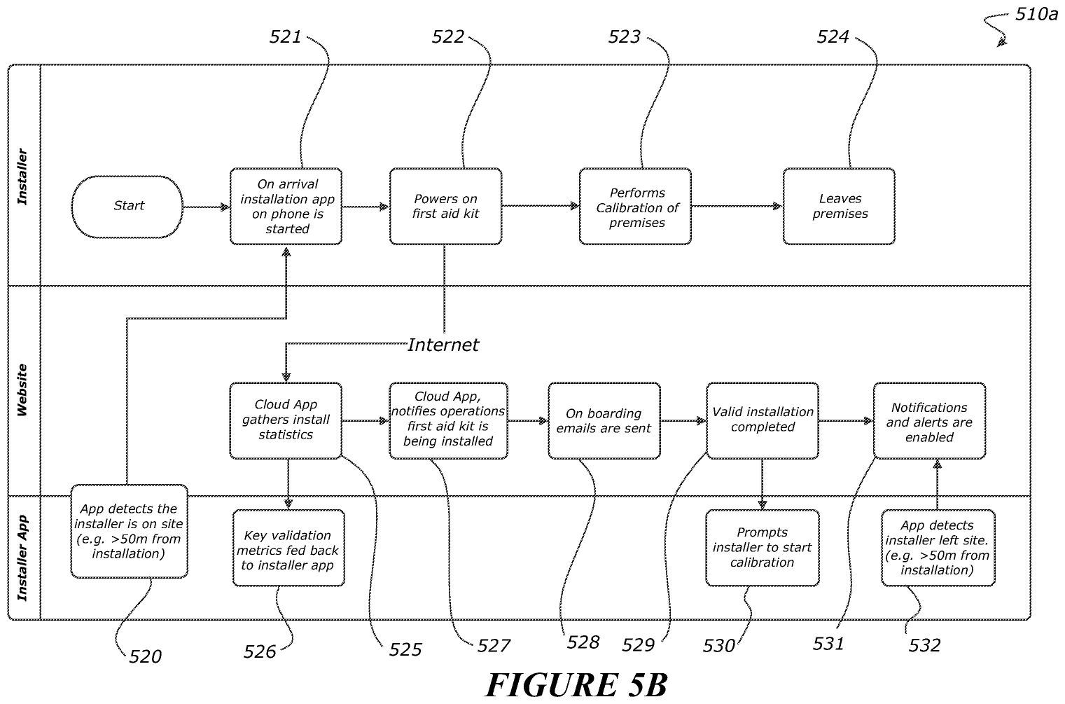

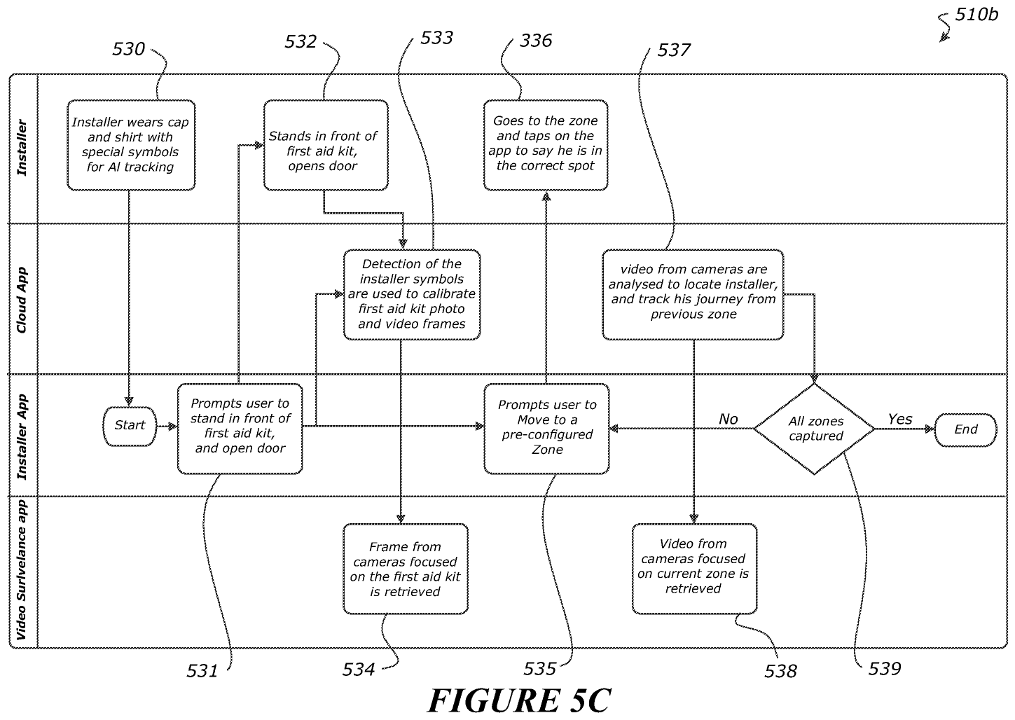

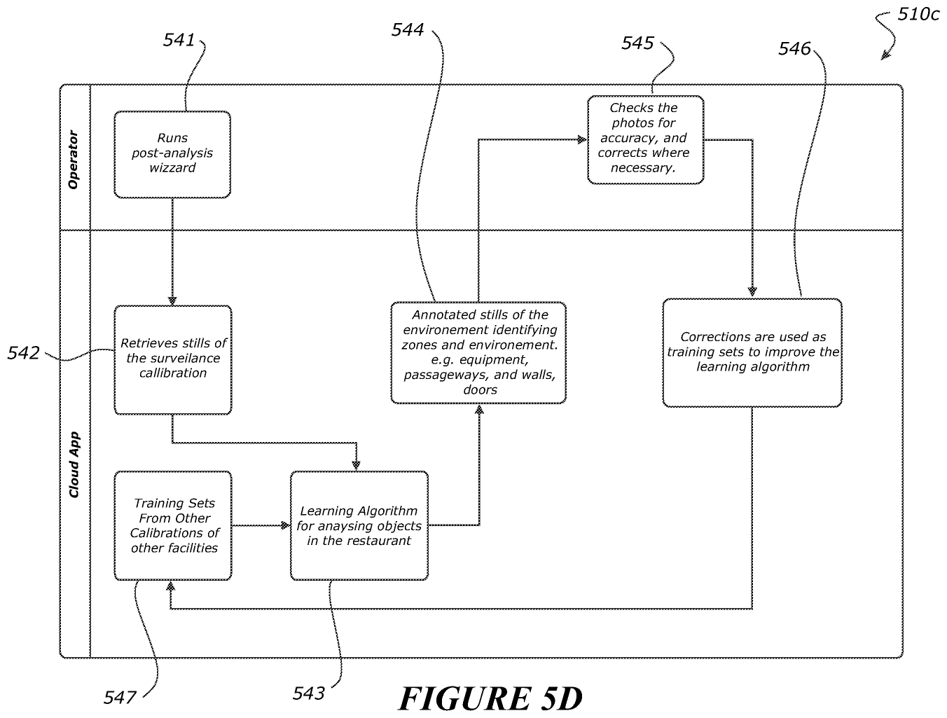

FIGS. 5A-5D illustrate an example of training and calibration of the machine learning algorithm for new installations. It may be appreciated that, as similar installations are installed, the amount of calibration needed may be reduced.

As shown in the flow diagram of FIG. 5A, the installation and calibration 500 of the health and safety management system 100 may comprise at least three steps. The first step 510a may involve installing the first aid kit 102. The second step 510b may involve calibrating the space and the third step 510c may involve carrying out post calibration.

An example of a working principle for the first step 510a which relates to installing the first aid kit 102 is illustrated in the flow diagram of FIG. 5B.

An application software (installer app) may detect that the installer is on the site (step 520). When the installer arrives at the site, the installer app on the device (e.g. phone) may be started (step 521). The installer may then power on the first aid kit 102 (step 522), perform the calibration (step 523) and leave the premises after the installation has been completed (step 524).

When the installer powers on the first aid kit 102 (step 522), the cloud application may gather statistics relating to the installation/install statistics (step 525) and key validation metrics may be fed back to the installer app (step 526). The cloud app may notify operations or any interested party that the first aid kit 102 is being installed (step 527), onboarding emails may be sent (step 528). When the valid installation is completed (step 529) the installer app may prompt the installer to start calibration (step 530). Notifications and alerts may be enabled (step 531). The installer app may detect that the installer has left the site (step 532) and that may cause step 531 to be performed.

Similarly, an example of a working principle for the second step 510b relating to calibrating the space is illustrated in the flow diagram of FIG. 5C.

The installer may wear a cap and shirt with special symbols for AI tracking (step 530). The installer app may prompt the user (installer in this case) to stand in front of the first aid kit 102 and open the door 207 (step 531). The installer may stand in front of the first aid kit 102 and open the door 207 (step 532) and the cloud application may allow detection of the installer symbols which may then be used to calibrate the first aid photo and video frames (step 533). The frames from the cameras focused on the first aid kit 102 may be retrieved through the video surveillance application software/video surveillance app (step 534). After step 531, the user (installer) may also be prompted to move to a pre-configured zone through the installer app (step 535) and the installer may then go to the zone and tap/send input command on the installer app to communicate that the installer is in the correct spot (step 536).

The cloud application may also allow video from cameras to be analysed to locate the installer and track the journey of the installer from a previous zone (step 537) and the video from cameras focused on a current zone may be retrieved using the video surveillance app (step 538).

After the video from cameras are analysed and the journey of the installer is tracked (i.e. after step 537), a check may be performed using the installer app to see if all zones are captured (step 539) and if not the process may revert back to the step of prompting user (installer) to move to a pre-configured zone (i.e. to step 535). If all zones are captured, then the process may end.

An example of a working principle for the third step 510c relating to post-calibration is explained in the flow diagram of FIG. 5D.

As shown, the operator may run a post analysis wizard (step 541), the cloud application may retrieve still images of the surveillance calibration (step 542). The cloud application may use machine learning algorithm to analyse objects in the premises, e.g. restaurant (step 543). The machine learning algorithm may be fed with data from step 542. Annotated stills of the environment identifying zones and environment (e.g. equipment, passageways, walls, doors etc) may be produced using cloud application (step 544). The operator may then check the still images/photos for accuracy and perform correction where necessary (step 545). Using cloud application, the correction may then be used as training sets to improve the machine learning algorithm (step 546). Using cloud application, data from step 546 may be added to training sets from other calibrations of other facilities (step 547). The data from step 547 to the machine learning algorithm of step 543.

The environment sensed by the machine learning algorithm at the third step 510c, i.e. the post-calibration step may be revisited as new hazards are identified. For example, if a manager notes that the tiles of a certain type are slippery under certain conditions, they may highlight that in the health and safety application, which may then search for other areas in other sites where the hazard may occur. If any site(s) where the hazard may occur is/are identified, then the health and safety application may alert the manager(s) of the respective site or sites to be aware of the potential hazard. The manager may then validate the hazard as true (i.e. real) or false and thereby allowing the learning algorithm that searches for the hazard to get more accurate. The hazard may also become part of the second step 510c, i.e. the step of calibrating the space. For example, the installer may be asked to visit spots in a site or sites that have a particular hazard, such as a tile type, and capturing/entering these locations on the health and safety application which may be installed in the computer(e.g. smartphone, tablet, PC, laptop, PDA etc.) of the installer.

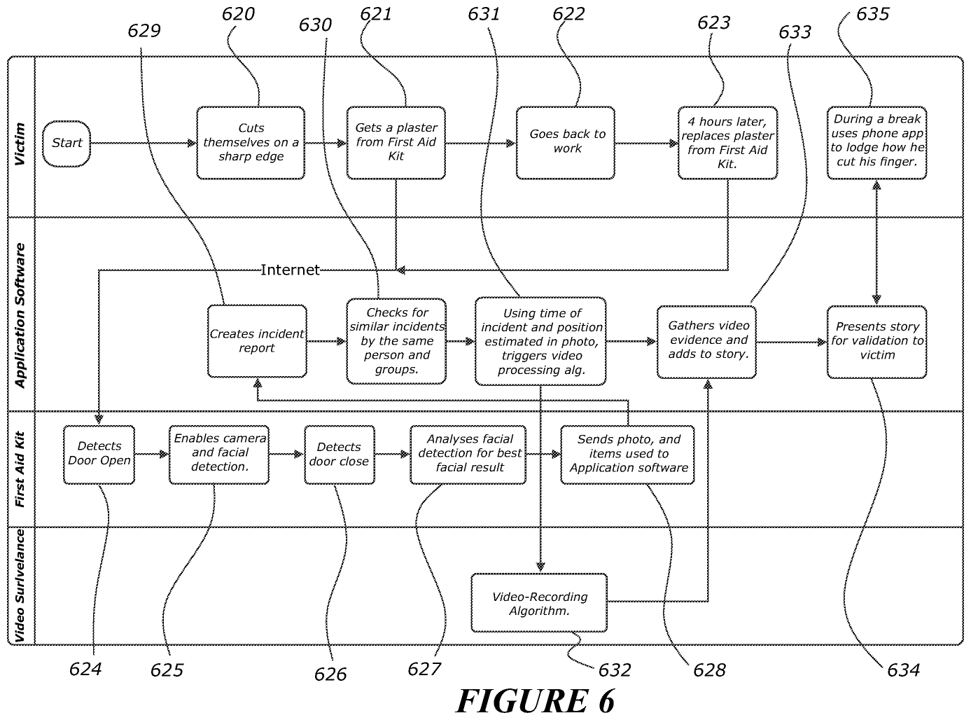

FIG. 6 is a flow diagram illustrating an example of the functionality of the health and safety management system 100 when an incident occurs. FIG. 6 is self-explanatory. In the example of FIG. 6, a person injures themselves, which in this example is a cutting of their finger on a sharp edge (step 620). That person then gets a plaster from first aid kit 102 (step 621) and then goes back to work (step 622). 4 hours later the plaster is replaced from the first aid kit 102 (step 623).

When the person gets a plaster from the first aid kit 102 (step 621) and replaces the plaster in the first aid kit (step 623), door 207 of the first aid kit 102 needs to be opened. When the door 207 is opened, the door opening is detected (step 624), camera 205 may be enabled and facial detection may be carried out (step 625). When the door is closed, the door closing is detected (step 626). The first aid kit may then analyse facial detection for the best facial result (step 627) and send photo and information regarding the items used using the application software (step 628). The application software may then create the incident report (step 629) and check for similar incidents by the same person and group, e.g. using historical data, (step 630). Using the time of incident and position estimated in photo, the application software may trigger the video processing algorithm (step 631) which may then be used with the video recording algorithm (step 632) to gather video evidence and is added to the story (step 633). After step 633 is performed, the story may be then presented to the victim (injured person) for validation (step 634). The victim may also add to the story by manually inputting information regarding the cause of the injury, which in this example is cutting of the finger, using input devices , e.g. smartphone (step 635).

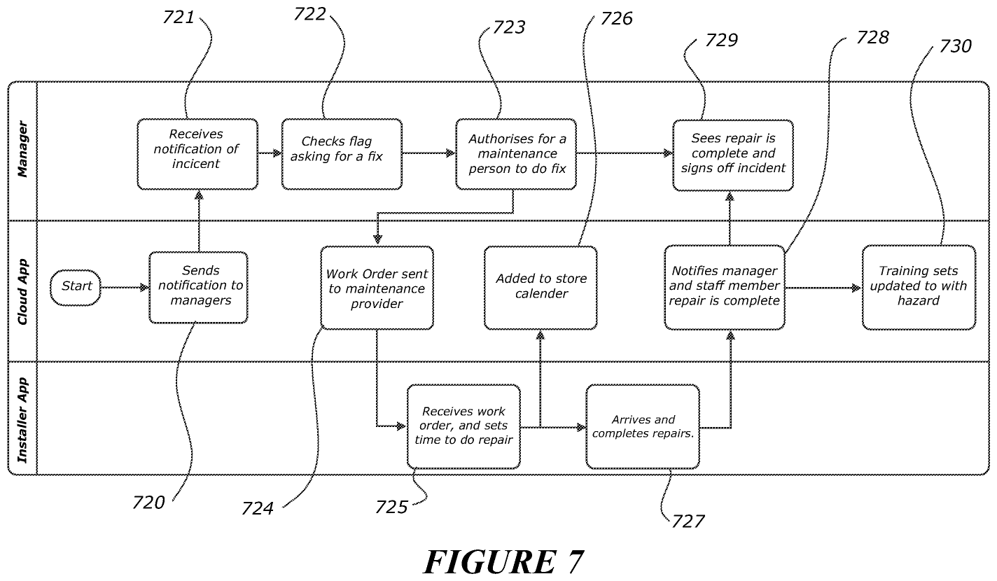

FIG. 7 is a flow diagram illustrating an example of how the health and safety management system 100 may allow interaction between the manager and a repair person (maintenance person) when the manager is notified of the incident that needs fixing. FIG. 7 is also self-explanatory. As shown in the example of FIG. 7, a notification may be sent to managers using the cloud application (step 720). When the relevant manager within the business receives a notification of the incident (step 721), the manager may then check the flag/send an input command asking for a fix (step 722) and may then authorise the repair person to do the fix (step 723). A work order may then be sent to the maintenance provider/person using the cloud application (step 724). The maintenance person/provider (installer) may receive the work order and set time to do repair using the application software referred to as the installer application (step 725). This time may be added to the calendar of the store where the manager is based using the communication between the installer application and the cloud application (step 726). At some stage, the repair person may arrive and complete the repair (step 727). The manager and staff member may be notified when the repair is completed using the cloud application (step 728). When the manager sees that the repair is completed, the manager may then sign off the incident (step 729). After notifying the manager and staff member that the repair is complete, the training set may be updated with data relating to the hazard (step 730).

Reverting to the example as shown in FIG. 3, where it is detected that plaster has been taken from the first aid kit 102. A machine learning algorithm of the health and safety management system 100 may be used to detect a staff member involved (e.g. a staff member involved in taking the plaster from the first aid kit 102, which could be the person injured). Three buttons 301a, 301b, 301c may be populated based on the plaster being only item taken, these three buttons are shown in FIG. 3 as ‘Start of shift’ button 301, a ‘New Injury’ button 301b and a ‘Replace old’ 301c.

If the photo 305a of the person taking the plaster matches with an earlier photo, i.e. if it is the same person taking another plaster, the two plaster takes may be combined, and the second take may be marked as a re-cover. The person (e.g. staff member) who triages the incident may accept that or alter it, e.g. if it is two injuries, it can be split apart and treated as two injuries.

For new injuries, the health and safety management system 100 may add in the video feeds, and the health and safety application may be updated with a possible zone the injury was incurred in.

Instead of typing, the person triaging the incident may use a speech recording ability using the speech record button 311a. The person triaging the incident may also add in photos (e.g. photos of the sharp edge that they cut their finger on) and click on needs fixing button (not shown) or the ‘Flag incident’ button 315a to alert the health and safety officer or manager or any other authorised person that there is a risk of others injuring themselves.

The manager may receive an alert after a maintenance person has finished. The alert may be generated using an alert generator. The manager may then check the work and sign off the incident. The original staff member involved may receive a notification that the incident has been fixed.

It can be appreciated that the health and safety management system 100 of the present invention may reduce harm by promoting actions to mitigate the causes of incidents.
Actions may be formed by learning from other incidents from other sites of a similar nature and presenting these to other sites, as preventative measures and when similar accidents/incidents occur.
By using the metric analysis algorithms, and presenting these to the manager for consideration, opportunities for risk elimination, mitigation or reduction may be identified and actioned.

The health and safety management system 100 may create actions for the managers in priority order. The health and safety management system 100 may even complete some of the actions automatically, e.g. sending training when particular things are detected.

The health and safety management system 100 may also present the area(s) that may need focusing. For example, reducing harm may be achieved by showing the manager where to focus their effort-e.g. training, environment or process change. A continuous improvement and feedback loop using machine learning algorithms across many sites utilising shared data may be used to answer various questions such as but not limited to:
What is the probability of the accident and where will it be?
What is the next action in order of priority to lower risk?
Where are the risks and how can they be mitigated?
What is the cost and risk reduction of the mitigation?
Recommendations on changes to operational processes

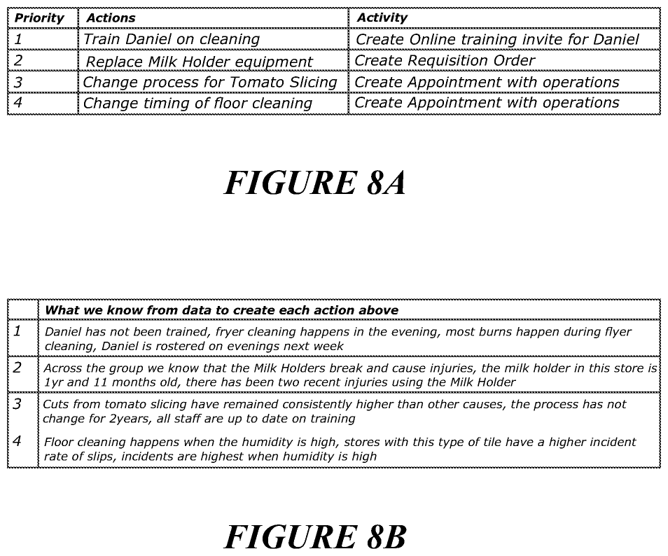

FIG. 8A shows an example of a list of some actions and corresponding semi-automated resolutions that may be presented to the manager, and FIG. 8B shows a step in the logic by which the health and safety management system 100 may generate the action.

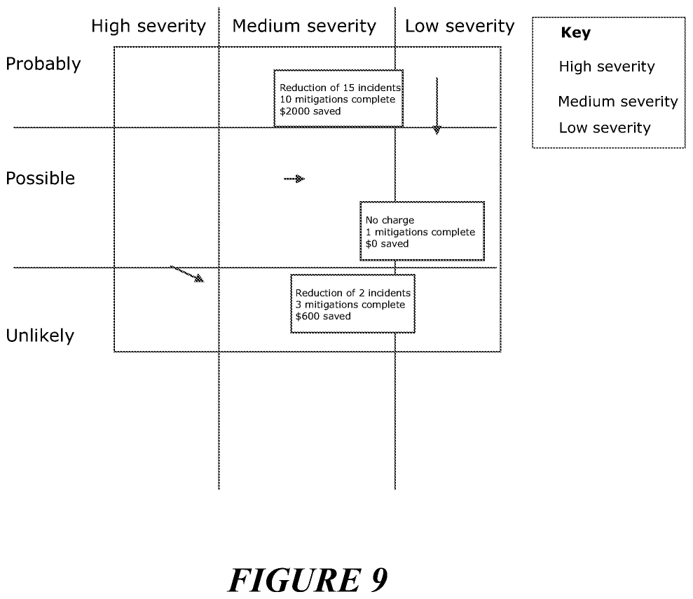

From the data, visualisation of the progress of health and safety could be represented in many forms. One of such forms is a Risk or Hazard matrix as shown in FIG. 9.

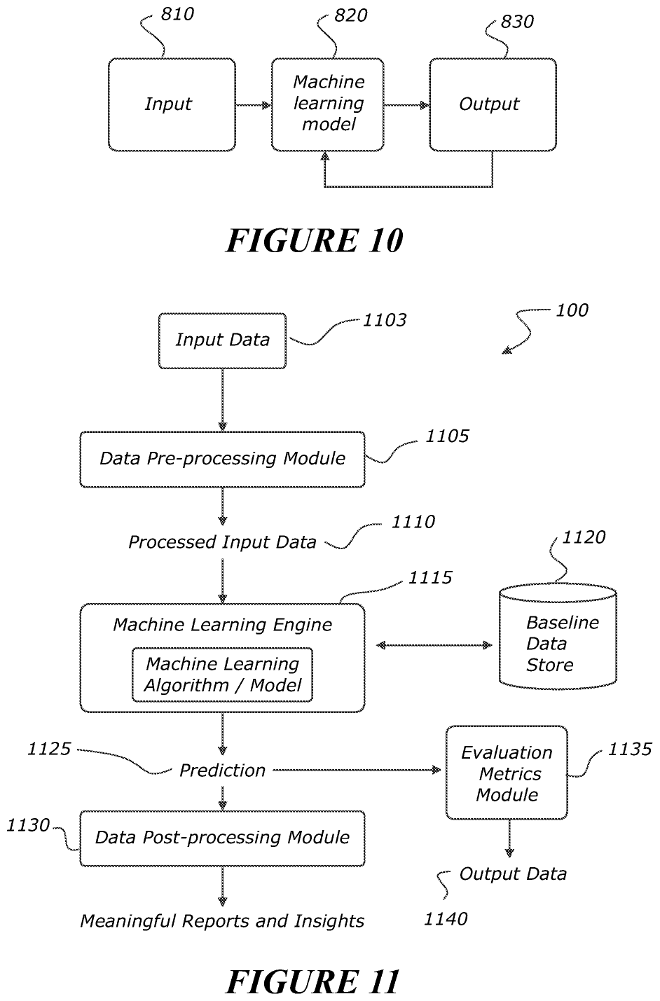

FIG. 10 is a schematic flow diagram illustrating one example of a working principle for a machine learning model that may be used in the health and safety management system 100 of the present invention.

As shown input 810 from one or more sources (e.g. cameras, environmental sensors, input devices, first aid kit, time clocks, transaction tills etc.) may be given to the machine learning model 820 which may then give an output 830 according to the algorithm that is applied, if the output 830 is right the output may be taken as the final result, else the feedback may be provided to the machine learning model 820 and the machine learning model 820 may be asked to predict until the machine learning model learns.

FIG. 11 is a schematic flow diagram showing one example of hardware implementation and working principle of the health and safety management system 100 of the present invention as described above for providing meaningful reports and insights.

As shown, the health and safety management system 100 may comprise a data pre-processing module 1105. The data pre-processing module 1105 may be configured to receive one or more inputs 1103 from one or more sources of health and safety related information, such as but not limited to cameras, environmental sensors, input devices, first aid kit, time clocks, transaction tills etc as described above. The data pre-processing module 1105 may comprise storage means (e.g. memory) for storing input data. The data pre-processing module 1105 may receive the input data 1103 as raw data from one or more sources of health and safety related information, and may process the raw input data 1103 by consolidating, analysing and/or interpreting the raw input data 1103 to provide the processed input data 1110. The processed input data 1110 may also include test and training set data. The processed input data 1110 may also include validation set data. The processed input data 1110 may then be fed into the machine learning engine 1115. The machine learning engine 1115 may be programmed or otherwise configured to perform machine learning algorithm/machine learning model analysis. The machine learning engine 1115 may comprise suitable hardware such as but not limited to Central Processing Unit (CPU) and/or Graphic processing Unit (GPU) to perform machine learning algorithm/machine learning model analysis.

The machine learning engine 1115 may comprise or may be operatively connected to at least one baseline data store 1120 which may be stored in a server, e.g. a cloud server. The baseline data store 1120 may store historical data relating to health and safety incidents. The historical data may be obtained from various sources including external sources. It may be appreciated that Application programming interfaces (APIs) or any suitable means or processes may be used for obtaining/gathering the historical data stored in the baseline data store 1120.

The machine learning model/algorithm may compare the processed input data with the data stored in the baseline data store to provide prediction 1125. The health and safety management system 100 may comprise a data post-data processing module 1130 that is programmed or otherwise configured to receive the prediction 1125. The data post-processing module 1130 may be programmed or otherwise configured to provide meaningful reports and insights regarding the health and safety at the worksite or a hospitality premise, which in this example is a restaurant.

The health and safety management system 1100 may also comprise an evaluation metrics module 1135 which may be that may be programmed or otherwise configured to receive the prediction 1125 and provide an output data 1140. That output data 1140 may be used for monitoring and/or analysing results from the machine learning engine 1115. Based on the output data 1140, feedback or correction signal may be sent to the machine learning engine 1115 to further improve the machine learning algorithm.

A skilled person will appreciate that the machine learning engine 1115 may be programmed or otherwise configured to split the processed input data 1110 (i.e. processed input dataset) into at least two parts, i.e. training set and testing set. Optionally, the machine learning engine 1115 may also split the processed input data into a validation set. The processed input data 1110 may be trained (and optionally validated) and may be tested using the testing set. The machine learning algorithm/model may be trained only using the training data set. The model may be validated by running the validation data set as needed. The accuracy of the test that is obtained from the test may indicate the accuracy that the machine learning algorithm/model may be expected to have when deployed.

It may be appreciated that the health and safety management system 100 described with reference to FIG. 11 may be used to provide all the functionalities as described above with reference to FIGS. 1 to 10. If needed, additional modules may be added or operationally communicated with the respective hardware of the health and safety management system to provide such functionalities.

In certain embodiments, one or more components (e.g. data post-processing module 1115, machine learning engine 1115 etc.) of the health and safety management system 100 as shown in FIG. 1110 may also be programmed and/or otherwise configured to send any notifications and alerts relating to prediction 1125 to one or more computers. Similarly, one or more components of the health and safety management system 100 (e.g. data pre-processing module 1105, machine learning engine 1115 etc.) as shown in FIG. 11 may also be programmed and/or otherwise configured to receive any notifications and alerts as notifications regarding prediction 1125 and/or alerts to computers which may include input devices, as explained above.

From the above descriptions with reference to FIGS. 1-11, it can be appreciated that the health and safety management system 100 of the present invention may provide at least the following features:
Capturing of first aid incidents that trigger a number of automated algorithms and learning-algorithms which further populate data into an incident report thereby reducing the process of form filling.
The use of other instigators, such as alarms, Personal Computers (PCs), applications on mobile phone/tablet may also be used to commence the gathering exercise, where for example a person sees any hazard that they consider necessary to be reported.

Incident reporting may be simplified as the user is presented with pre-filled data, that can be accepted or altered. In most cases, the acceptance of the incident or annotation of data may be achieved by:
An easy to use dynamic interface that may adapt to incident data that may add a human element to the incident report with minimal effort.
Feedback from human validated incident reports may be used to train the health and safety management system, thereby improving the accuracy of captured and predicted information.
Metric analysis may use the enhanced incident reports and information from other sources, such as time records clocks, queue counters (cars in drive-through, customers being served, orders in progress, customers in the store, staff in the vicinity of accident) to remove confounding factors from the metric-analysis, allowing deeper insights into the root causation of safety issues.
Information from other similar sites may be used to mitigate hazards predictively and improve the training sets of all learning algorithms.
Suggested escalation of the incident based on similar incidents or knowledge of other mitigations may be automatically generated.

By capturing all or substantially all metrics, metrics generated may be complete data sets or substantially complete data sets that represent the safety of the site. The metrics may be based on factual captured data rather than opinions and recollected events. As such, the metrics may be used to determine the causality of risks that lead to the incidents and may predictively assist managers or the business to focus on effort on hazard reduction, e.g. training, hazard elimination, mitigation or reductions.

Although the foregoing describes several embodiments of the present invention with reference to restaurants, the foregoing may equally apply to any other suitable worksite(s) or hospitality premises that is/are not necessarily restaurants.

Thus, it can be appreciated that in one aspect of the present invention may include a health and safety management system for a worksite or a business or a hospitality premises which comprises at least one source 102, 104, 106, 108a, 108b, 108c, 110, 112, 115, 116 of health and safety related information of the hospitality premises. The system 100 further comprises at least one machine learning engine 1115 that may be configured to receive the health and safety related information as at least one input data 1103. The machine learning engine may be further configured for analysing the input data 1103 and predict at least one issue relating to health and safety at the hospitality premises that requires attention by at least one authorised party. The machine learning engine may use a machine learning algorithm for analysing the input data 1103 and predict at least one issue relating to health and safety at the hospitality premises that requires attention by at least one authorised party. The authorised party may be any authorised person or organisation e.g. manager(s), government regulatory body(ies), staff member(s) etc.

Another aspect of the present invention may include a health and safety management system 1110 for a worksite (i.e. a business premises). The health and safety management system comprises at least one source of health and safety related information of the worksite. That source of health and safety related information is or comprise a monitored dispensary apparatus 102. The monitored dispensary apparatus 102 to which multiple users have access. The monitored dispensary apparatus has a containment region inside of/within which items are stored. The items contain or are items for use in medical treatment. The monitoring dispensary apparatus comprises or is in an operative communication with a monitoring system comprising at least one detector (e.g. camera 205) (i.e. preferably located inside/within the containment region) that is able to detect any use of the monitored dispensary apparatus 102. An output of the detector(s) may be the health and safety information of the worksite. The health and safety management system 100 further comprises at least one machine learning engine 1115 that is configured to receive the health and safety related information as at least one input data 1103 and may be further configured to analyse the input data 1103 and predict at least one issue relating to health and safety at the workplace that requires attention by at least one authorised party. The machine learning engine may be configured to use a machine learning algorithm for analysing the input data 1103 and predict at least one issue relating to health and safety at the workplace/worksite that requires attention by at least one authorised party. The authorised party may be any authorised person or organisation e.g. manager(s), government regulatory body(ies), staff member(s) etc.

The monitored dispensary apparatus 102 may be a first aid kit or a first aid station. At least one source may be selected from a smartphone, laptop, personal computer (PC), tablet or PDA.

The health and safety management system may further comprise at least one computer having a GUI. The GUI may be adapted to access the health and safety related information and/or prediction. The GUI may be operative on at least one of a display screen of the computer. The computer may be the input device 104, 106, 110, 112. The camera(s) 108a, 108b, 108c may be video camera(s) and/or a surveillance camera(s). The health and safety management system may further comprise an alert generator for generating and sending an alert to at least one authorised party regarding the prediction. The GUI may incorporate randomising and changing the order of buttons or randomising and changing one or more button phrasing to detect/avoid giving false information. For example, if the user(s) are not paying attention and are rushing, randomising and changing the order of buttons or randomising and changing one or more button phrasing may occur to detect/avoid giving false information. This may be achieved using standard survey and statistical gathering tools.

The machine learning engine(s) 1115 may be configured to generate the prediction 1125 as an output data (output information) for consideration by said at least one authorised party. The one machine learning engine(s) 1115 may be configured to generate the prediction 1125 as output data (output information) for monitoring and/or analysing results.

The health and safety management system may further comprise a first data processing module (data pre-processing module 1105) that may be configured to receive the health and safety related information from the source(s) 102, 104, 106, 108a, 108b, 108c, 110, 112, 115, 116 as an input and process the input as a processed health and safety information (processed input data 1110). The machine learning engine(s) 1115 may be configured to receive the processed health and safety information as at least one input data 1103.

The health and safety management system 100 may be configured to generate a report and insights regarding the health and safety at the hospitality premises or the worksite.

The health and safety management system 100 may further comprise a second data processing module (data post-processing module 1130) that may be configured to receive the prediction 1125 and generate the report and insights regarding the health and safety at the hospitality premises or the worksite based on the prediction.

The health and safety management system 100 may further comprise an evaluation metrics module 1135 that may be configured to receive the prediction 1125 and output data 1140 for monitoring and/or analysing results from the machine learning engine(s) 1115.

In certain embodiments, the data pre-processing module 1105, the data post processing module 1130 and/or the evaluation metrics module 1135 may be part of the machine learning engine(s) 1115.

The machine learning engine(s) 1115 may be programmed or configured to split the processed input data 1110 into at least two parts namely a training set and testing set. The machine learning engine(s) 1115 may be further programmed to split the processed input data into a validation set.

The worksite or hospitality premises may be a restaurant, e.g. a quick service restaurant.

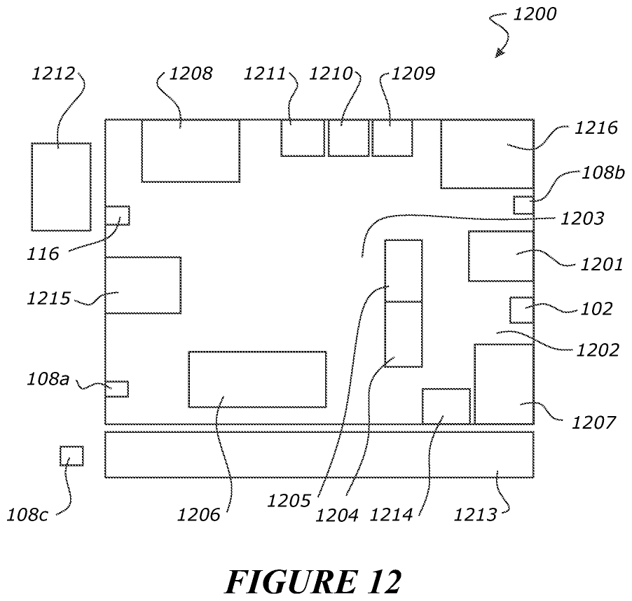

FIG. 12 is a schematic diagram showing an example of a layout of a worksite or a hospitality or a business premises which in this example is a restaurant 1200 that may be implemented in the health and safety management system of FIG. 1. As shown the restaurant may include a kitchen or food cooking facility 1201, a food preparation area 1202, a food order area 1203, a payment transaction area 1204, a food service area 1205, a dining area 1206 (for customers), a food storage area 1207 (e.g. an area containing a fridge or a freezer or cupboards or shelves), a food dispensing area 1208 (e.g. drink and condiment dispensing area), a wash-up area 1209, a washroom area 1210, a toilet area 1211, kids play area 1212, a drive-through area 1213, a drive-through payment transaction area 1214, an entrance area, and an office/staffroom area 1216. Possible locations of some of the sources of health and safety information such as first aid kit 102, cameras 108a, 108b, 108c and moisture sensors 116 as described above are also shown.

Of course, the layout of restaurant 1200 may be different to what is shown in the example of FIG. 12. Also, restaurant 1200 may contain additional area(s) or less area(s) than what is shown in FIG. 12. Similarly, restaurant 1200 may contain additional or less source(s) of health and safety information or less area(s) than what is shown in FIG. 12.

A hospitality premises health and safety management method according to one aspect of the present invention may comprise at least the following steps:
receiving at least one source of health and safety related information of the hospitality premises from at least one source of health and safety related information of the hospitality premises;
feeding the health and safety related information as at least one input data to at least one machine learning engine;
using the machine learning engine for analysing the at least one input data; and
making a prediction of at least one issue relating to health and safety at the hospitality premises that requires attention by at least one authorised party.

Similarly, a health and safety management method for a worksite (i.e. a business premises) according to one aspect of the present invention may comprise at least the following steps:
receiving at least one source of health and safety related information of the worksite from at least one source of health and safety related information of the worksite, the at least one source of information comprising or being a monitored dispensary apparatus 102 to which multiple users have access, the monitored dispensary apparatus comprising:
   a containment region inside of/within which items are stored, wherein the items contain or are items for use in medical treatment,
   wherein the monitored dispensary apparatus further comprises or is in operative communication with a monitoring system,
   wherein,
   wherein the monitored dispensary apparatus comprises or is in operative communication with the monitoring system comprising at least one detector (e.g. camera 205) located (e.g. preferably inside the containment region) that is able to detect any use of the monitored dispensary apparatus, and
wherein an output of the one or more detectors is the health and safety information of the worksite; and
   feeding the health and safety related information as at least one input data to at least one machine learning engine that is configured to implement a machine learning algorithm.

The machine learning engine may be configured to implement a machine learning algorithm and the method may comprise:
using said machine learning algorithm for analysing at least one input data and for making predictions.

Methods and processes described herein may be embodied in, and partially or fully automated via, software module(s) executed by one or more general and/or special purpose computers. Software module(s) can refer to logic(s) embodied in hardware and/or firmware, or to a collection of software instructions, possibly having entry and exit points, written in a programming language, such as, for example, C or C++. A software module(s) may be compiled and linked into an executable program, installed in a dynamically linked library, or may be written in an interpreted programming language such as, for example, R, BASIC, Perl, or Python. It will be appreciated that software modules may be callable from other software modules or from themselves, and/or may be invoked in response to detected events or interrupts. Software instructions may be embedded in firmware, such as an erasable programmable read-only memory (EPROM). It will be further appreciated that hardware modules may comprise connected logic units, such as gates and flip-flops, and/or may comprised programmable units, such as programmable gate arrays, application specific integrated circuits, and/or processors. The modules described herein can be implemented as software modules, but also may be represented in hardware and/or firmware. Moreover, although in some embodiments a module may be separately compiled, in other embodiments a module may represent a subset of instructions of a separately compiled program and may not have an interface available to other logical program units.

In certain embodiments, software modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with users, operators, other systems, components, programs, and so forth.

Where in the foregoing description reference has been made to elements or integers having known equivalents, then such equivalents are included as if they were individually set forth.

Although the invention has been described by way of example and with reference to particular embodiments, it is to be understood that modifications and/or improvements may be made without departing from the scope or spirit of the invention.

1. A health and safety management system for a worksite, said health and safety management system comprising:
at least one source of health and safety related information of said worksite, said at least one source of health and safety related information comprises or is a monitored dispensary apparatus to which multiple users have access, said monitored dispensary apparatus comprising a containment region inside of which items are stored, said items contain or are items for use in medical treatment,
wherein the monitored dispensary apparatus further comprises or is in operative communication with a monitoring system that comprises at least one detector that is configured to detect any use of said monitored dispensary apparatus,
wherein, an output of said at least one detector is said health and safety related information of said worksite, and
wherein, the said health and safety management system further comprises at least one machine learning engine that is configured to receive said health and safety related information as at least one input data and is further configured to analyse said at least one input data and make a prediction of at least one issue relating to health and safety at said workplace that requires attention by at least one authorised party.

2. The health and safety management system as claimed in claim 1, wherein said at least one detector is located within said containment region.

3. The health and safety management system as claimed in claim 1, wherein said machine learning engine is configured to use a machine learning algorithm for analysing said at least one input data and make said prediction of said at least one issue relating to health and safety at said workplace that requires attention by said at least one authorised party.

4. The health and safety management system as claimed in claim 1, wherein said at least one source is configured to detect or capture a health and safety related incident, said health and safety related incident being said health and safety related information.

5. The health and safety management system as claimed in claim 1, wherein said monitored dispensary apparatus is a first aid kit or a first aid station.

6. The health and safety management system as claimed in claim 1, wherein said at least one source comprises or functions as at least one input device that is a computer and that allows a user to manually input said health and safety related information.

7. The health and safety management system as claimed in claim 1, wherein said health and safety management system further comprises at least one electronic device having a display screen and a graphical user interface (GUI) displayed in said display screen, wherein said GUI is adapted to access said health and safety related information and/or said prediction, wherein said GUI is operative on said display screen.

8. The health and safety management system as claimed in claim 7, wherein said at least one electronic device is said at least one input device.

9. The health and safety management system as claimed in claim 1, wherein said health and safety management system further comprises an alert generator for generating and sending an alert to said at least one authorised party regarding said prediction.

10. The health and safety management system as claimed in claim 1, wherein said health and safety management system further comprises a first data processing module that is a data pre-processing module that is configured to receive said health and safety related information from said at least one source as an input and process said input as a processed health and safety information.

11. The health and safety management system as claimed in claim 1, wherein said at least one machine learning engine is configured to receive said processed health and safety information as said at least one input data.

12. The health and safety management system as claimed in claim 1, wherein said at least one machine learning engine is configured to generate said prediction as an output data for consideration by said at least one authorised party.

13. The health and safety management system as claimed in claim 1, wherein said at least one machine learning engine is configured to generate said prediction as an output data for monitoring and/or analysing results from said at least one machine learning engine.

14. The health and safety management system as claimed in claim 1, wherein said health and safety management system is configured to generate a report and insights regarding said health and safety at said worksite.

15. The health and safety management system as claimed in claim 13, wherein said health and safety management system further comprises a second data processing module that is a data post-processing module configured to receive said prediction and generate said report and insights regarding said health and safety at said worksite based on said prediction.

16. The health and safety management system as claimed in claim 13, wherein said health and safety management system further comprises an evaluation metrics module that is configured to receive said prediction and output data for monitoring and/or analysing results from said at least one machine learning engine.

17.-21. (canceled)

22. A health and safety management method for a worksite, said health and safety management method comprising:
receiving at least one source of health and safety related information of said worksite from at least one source of health and safety related information of said worksite, said at least one source of information comprises or is a monitored dispensary apparatus to which multiple people have access, said monitored dispensary apparatus comprising:
a containment region inside of which items are stored, wherein said items contain or are items for use in medical treatment,
wherein the monitored dispensary apparatus further comprises or is in communication with a monitoring system that comprises at least one detector and is able to detect any use of said monitored dispensary apparatus, and
wherein an output of said one or more detectors is said health and safety information of said worksite, wherein the method further comprises:
feeding said health and safety related information as at least one input data to at least one machine learning engine, and
using the machine learning engine to analyse said at least one input data and make a prediction of at least one issue relating to health and safety at said workplace that requires attention by at least one authorised party.

23. The health and safety management method as claimed in claim 22, wherein said method further comprises generating and sending an alert to said at least one authorised party regarding said prediction.

24. The health and safety management method as claimed in claim 22, wherein said method further comprises receiving said health and safety related information from said at least one source as an input and process said input as a processed health and safety information.

25. (canceled)

26. The health and safety management method as claimed in claim 22, wherein the method further comprises generating a report and insights regarding said health and safety at said worksite and/or generating said prediction as an output data for consideration by said at least one authorised party.

27-36. (canceled)
